(19) United States
(12) Patent Application Publication
(10) Pub. No.: US 2013/0345249 A1
(43) Pub. Date: Dec. 26, 2013

(54) Formulations for the prevention and treatment of Wolbachia-related disease

(71) Applicants: Alain Debec, Paris (FR); Laura Serbus, Miami, FL (US); Frederic Landmann, Santa Cruz, CA (US); William Sullivan, Santa Cruz, CA (US)

(72) Inventors: Alain Debec, Paris (FR); Laura Serbus, Miami, FL (US); Frederic Landmann, Santa Cruz, CA (US); William Sullivan, Santa Cruz, CA (US)

(73) Assignee: The Regents of the University of California, Oakland, CA (US)

(21) Appl. No.: 13/979,841

(22) PCT Filed: Jan. 13, 2012
(86) PCT No.: PCT/US12/21252

§ 371 Date: Sep. 16, 2013

Related U.S. Application Data
(60) Provisional application No. 61/433,203, filed on Jan. 15, 2011.

(51) Int. Cl. A61K 31/136; A61K 31/05; A61K 31/4709; A61K 31/475

(52) U.S. Cl. CPC A61K 31/136; A61K 31/475; A61K 31/05; A61K 31/4709
USPC 514/280; 562/488; 546/171; 568/745; 546/55; 514/569; 514/736; 514/314

(57) ABSTRACT

The invention encompasses pharmaceutical formulations for the prevention and treatment of Wolbachia-related disease wherein the formulations comprise a compound previously unknown and unused for such a purpose, the compound being one or more of: Pararosaniline Pamoate, Pyrvinium Pamoate, Clofoctol, and Isoreserpine, derivatives, metabolites, precursors, pro-drugs and variants thereof.

### RELATIONSHIP TO OTHER APPLICATIONS

This application claims priority to and the benefit of U.S. provisional application No. 61433203 filed 15 Jan. 2011, which application is fully incorporated by reference for all purposes.

### FIELD OF THE INVENTION

The field of the invention encompasses prevention and treatment of Wolbachia-related disease and the use of various compounds including Pararosaniline, Pyrvinium, Pamoate, Clofoctol, and Isoreserpine to formulate pharmaceutical compounds that may be used to kill Wolbachia in vivo.

### BACKGROUND

A number of references are believed by the inventors to be particularly relevant to the present work and include those shown in the “References” section. These references are hereby incorporated by reference for all purposes. The fact that these references are cited is not an admission that they are prior art.

### BRIEF DESCRIPTION OF THE INVENTION

The invention encompasses pharmaceutical formulations for the prevention and treatment of Wolbachia-related disease. In particular the invention encompassed the use of various compounds that may be formulated so that they may be applied to a subject to kill Wolbachia in vivo. Compounds used in the formulations of the invention include Pararosaniline Pamoate, Pyrvinium Pamoate, Clofoctol, Isoreserpine and derivatives, metabolites, precursors, pro-drugs and variants thereof. The formulations may be administered to a subject suffering from (or in danger of suffering from) a Wolbachia-related disease either topically or systemically so as to kill Wolbachia. The formulations of the invention may comprise one or more of Pararosaniline Pamoate, Pyrvinium Pamoate, Clofoctol, and Isoreserpine, or any other compound identified by the screening method of the invention, either singly on in combination.

### BRIEF DESCRIPTION OF THE FIGURES

FIG. 1. Drosophila tissue culture imaged in a 384-well plate by automated microscopy. A) Wolbachia-infected cells. B) Identical cell line cured by a 4-week tetracycline treatment. Blue: cytoplasmic Wolbachia. Green: microtubules. Red: host nuclei.

FIG. 2. Overview of the chemical screen.

FIG. 3. Results from B. malayi drug feeding assay A.) Viable DMSO-treated worms. B.) Inviable pararosaniline pamoate-treated worms. C.) Timetable of worm mortality resulting from various treatments.

FIG. 4. D. melanogaster viability after consumption of pararosaniline pamoate at various concentrations.

FIG. 5. Wolbachia titer in Drosophila is reduced by treatment with pararosaniline pamoate. Graph shows average quantity of Wolbachia within a single focal plane of multiple D. melanogaster oocytes. n=number of oocytes analyzed.

### GENERAL REPRESENTATIONS CONCERNING THE DISCLOSURE

The embodiments disclosed in this specification are exemplary and do not limit the invention. Other embodiments can be utilized and changes can be made. As used in this specification, the singular forms “a”, “an”, and “the” include plural reference unless the context clearly dictates otherwise. Thus, for example, a reference to “a part” includes a plurality of such parts, and so forth. The term “comprises” and grammatical equivalents thereof are used in this specification to mean that, in addition to the features specifically identified, other features are optionally present. Where reference is made in this specification to a method comprising two or more defined steps, the defined steps can be carried out in any order or simultaneously (except where the context excludes that possibility), and the method can optionally include one or more other steps which are carried out before any of the defined steps, between two of the defined steps, or after all the defined steps (except where the context excludes that possibility). Where reference is made herein to “first” and “second” features, this is generally done for identification purposes; unless the context requires otherwise, the first and second features can be the same or different, and reference to a first feature does not mean that a second feature is necessarily present (though it may be present). Where reference is made herein to “a” or “an” feature, this includes the possibility that there are two or more such features. This specification incorporates by reference all documents referred to herein and all documents filed concurrently with this specification or filed previously in connection with this application, including but not limited to such documents which are open to public inspection with this specification.

### Definitions

The following words and phrases are used herein as follows:

The terms “pharmaceutical formulation” and “pharmaceutical composition” mean any composition intended for administration to a human being or other mammal and comprises at least one drug; it may also include one or more other additives, for example pharmaceutically acceptable excipients, carriers, penetration enhancers, stabilizers, buffers or other materials.

The term “drug” means any substance that alters the physiology of an organism. Multiple drugs may be included in a single formulation.

The term “therapeutically effective amount” means an amount of a therapeutic agent, or a rate of delivery of a therapeutic agent, effective to facilitate a desired therapeutic effect.

The term “treatment” means the application of a process to an individual in order to alter a physiological state, whether or not the process includes a curative element.

“Controlled” release of a drug means release of the drug in a pre-determined or adjustable way such that the amount or rate or timing of release is pre-set or is altered in a desired way.

“Sustained” release of a drug means release over an extended period of time, for example minutes, hours or days, such that less than all the drug is released initially.

The term “subject” means any subject, generally a mammal (e.g., human, primate, canine, feline, equine, bovine, fish, birds etc in which management of a disease is desired.

### DETAILED DESCRIPTION OF THE INVENTION

The invention encompasses pharmaceutical formulations for the prevention and treatment of Wolbachia-related disease. In particular the invention encompasses pharmaceutical formulations containing a drug that kills Wolbachia in vivo in a treated subject. The invention includes the use of various compounds (drugs) that may be formulated into a pharmaceutical formulations so that on administration to a subject they kill Wolbachia in vivo. Compounds used in the formulations of the invention include Pararosaniline Pamoate, Pyrvinium Pamoate, Clofoctol, and Isoreserpine and derivatives, metabolites, and precursors, pro-drugs and variants thereof. The formulations may be administered to a subject suffering from (or in danger of suffering from) a Wolbachia-related disease either topically or systemically so as to kill Wolbachia.

It is believed that that (and a non-exhaustive search of PubMed and the Delphion patent database confirms) that the named drugs (Pararosaniline Pamoate, Pyrvinium Pamoate, Clofoctol, and Isoreserpine) have not previously been used to treat Wolbachia-related disease. Thus the invention includes new uses for known compounds and methods for treatment, wherein Pararosaniline Pamoate, Pyrvinium Pamoate, Clofoctol, and Isoreserpine and derivatives, metabolites, and precursors, pro-drugs and variants thereof are used to prevent and treat Wolbachia-related disease. The invention also includes methods for using he same compounds and formulations to treat other filarial diseases.

The formulations of the invention may also include one or more other additives, for example pharmaceutically acceptable excipients, carriers, penetration enhancers, stabilizers, buffers or other materials.

The formulations of the invention may be administered in a number of ways including via inhalation; or topically, e.g., onto the skin or as eye drops or ear drops; or as a pessary or suppository, or enterally, e.g., orally, parenterally, e.g., intravenously, intramuscularly etc.

The invention encompasses use of the pharmaceutical formulations described to treat or prevent Wolbachia-related disease. Such uses include the use of one or more compounds, either signally or in combination, selected from the group consisting of: Pararosaniline Pamoate, Pyrvinium Pamoate, Clofoctol, and Isoreserpine, and derivatives, metabolites, precursors, prodrugs and variants thereof.

The invention encompasses methods for treating of preventing Wolbachia-related disease wherein the method comprises the following steps: (1) providing am anti-Wolbachia formulation wherein the formulation comprises at least one compound selected from the group consisting of Pararosaniline Pamoate, Pyrvinium Pamoate, Clofoctol, and Isoreserpine, and derivatives, metabolites, precursors, pro-drugs and variants thereof. (2) providing a subject in need of treatment or prevention of a Wolbachia-related disease; (3) providing to the subject a therapeutically effective amount of the anti-Wolbachia formulation by various means including (but not limited to) orally, intravenously, intramuscularly, by inhalation, or topically, either in a bolus or a sustained release form. Such treatment results in the measurable killing of Wolbachia in vivo.

The methods of treatment may be used to treat mammals including humans, pets such as dogs and cats, horses, domestic livestock, ungulates, bovines, ovines, foul, fish etc.

The invention encompasses methods for killing Wolbachia in vitro and in vivo, and includes methods wherein the degree of killing of Wolbachia is measured experimentally using methods described herein. One methods involves the following steps: (1) performing a feeding assay using Wolbachia-infected Drosophila melanogaster flies; (2) quantitavely determining the treatment's effect of the quantity of Wolbachia carried by the D. melanogaster host.

In various embodiments using this method the quantity of viable Wolbachia organisms is reduced by at least 20%, or 30%, or 40%, or 50%, or 60% or 70% or at least 80% when compared to the pre-treatment level. In various clinical embodiments the quantity of viable Wolbachia organisms in vivo is reduced by a similar amount.

The invention encompasses the production and formulation of pharmaceutical formulations described to treat of prevent Wolbachia-related disease.

The invention encompasses methods for screening candidate compounds that may be used to kill Wolbachia in vivo. These methods are extensively described herein.

The invention encompasses a microscopy-based high throughput screening assay that identifies compounds that reduce Wolbachia titer in tissue culture cells.

### Investigational Protocols and Data

Wolbachia are intracellular bacteria that infect arthropods as well as nematodes that cause Elephantiasis and Onchocerciasis in millions of people. Anti-filarial drugs have had only limited success in combating these diseases (Hoerauf, 2008). Exciting recent discoveries showed that parasitic nematodes require Wolbachia for survival and these bacteria are causally involved in disease manifestation (Saint Andre et al., 2002; Turner et al., 2009).

Antibiotics can now be used to improve anti-filarial drug therapies (Hoerauf et al., 2008), but the treatments are expensive and require a minimum 4-6 week duration. To address these issues, we have developed and tested an approach to identify small molecule inhibitors that rapidly reduce Wolbachia quantity residing within eukaryotic cells. To identify these inhibitors, we treated Wolbachia-infected tissue culture cells with many different inhibitors. We identified the compounds that most reduced Wolbachia quantity within the cells, and then validated the efficacy of the compound in Wolbachia-infected organisms. Compounds that show efficacy in animal models are of significant interest, as they offer new options for fast-acting, Wolbachia-eliminating therapies.

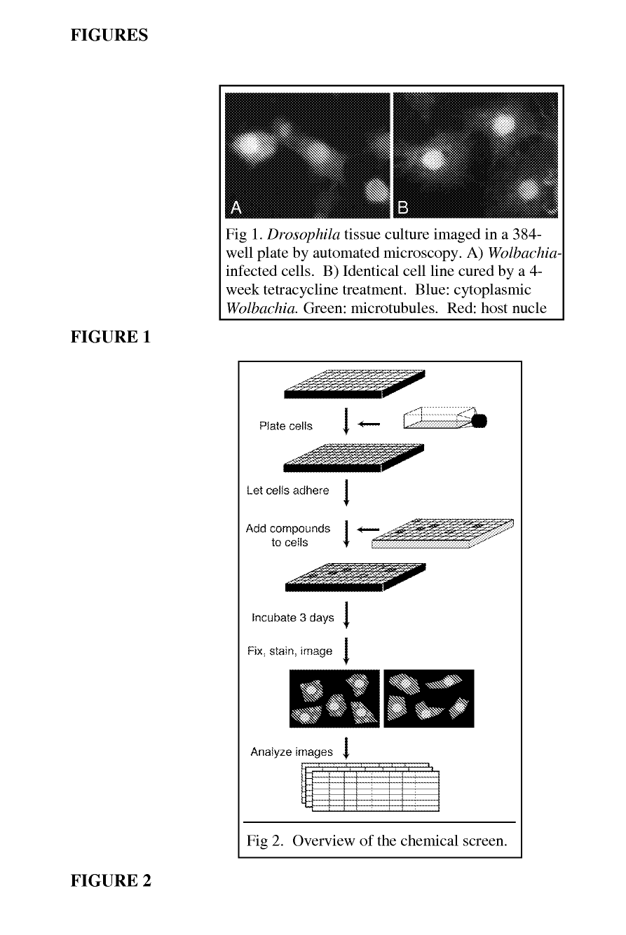

High-throughput screening is now well-established in the literature as a reliable technique (Carpenter, 2007; Perrimon and Mathey-Prevot, 2007). To conduct our screen, we first generated Drosophila tissue culture cells that are constitutively infected with Wolbachia as previously (Szollosi and Debec, 1980) (FIG. 1). These lines have stably held the Wolbachia infection since inception (3.5 years), and they exhibit no obvious defects in morphology or mitotic ability. Our cell lines additionally carry a GFP-Jupiter transgene that fluorescently labels microtubules (Karpova et al., 2006), thus clearly delineating cellular boundaries. We refined a seeding/fixation/staining protocol to enable clear detection of host nuclei and cytoplasmic Wolbachia using fluorescent probes. After adapting our protocols to a 384-well format, we validated them at the UCSC Chemical Screening Center using automated, programmable liquid handlers and an automated epifluorescence microscope (FIG. 2). In collaboration with the microscope company Molecular Devices, we optimized journaling software that analyzes the level of cytoplasmic DAPI (Wolbachia) fluorescence per cell in all wells of the plate. This journal reports that infected cells (FIG. 1A) on average contain six-fold more cytoplasmic DAPI than uninfected control cells (FIG. 1B), indicating that Wolbachia fluorescence is unambiguously detectable over background. Taken together, these steps set the stage for a pilot high-throughput screening test.

To test the premise that our assay will identify compounds useful for elimination of Wolbachia, we conducted a pilot screen of 2000 FDA—approved drugs from the Spectrum chemical library collection at the UCSC Chemical Screening Center. In this pilot, compounds were added to Wolbachia-infected cells at a final concentration of 100 uM in media and allowed to incubate for 3 days. Cells were then fixed, stained, imaged, and analyzed by automated machinery. Thus far 11 compounds were identified that reduced cytoplasmic DAPI fluorescence to the background level of uninfected control cells, suggesting that intracellular Wolbachia became depleted in the presence of those compounds. We examined the images by eye to verify the Wolbachia disruption and prioritized the drug candidates so those with the least overt impact on host cell morphology would be the first ones pursued.

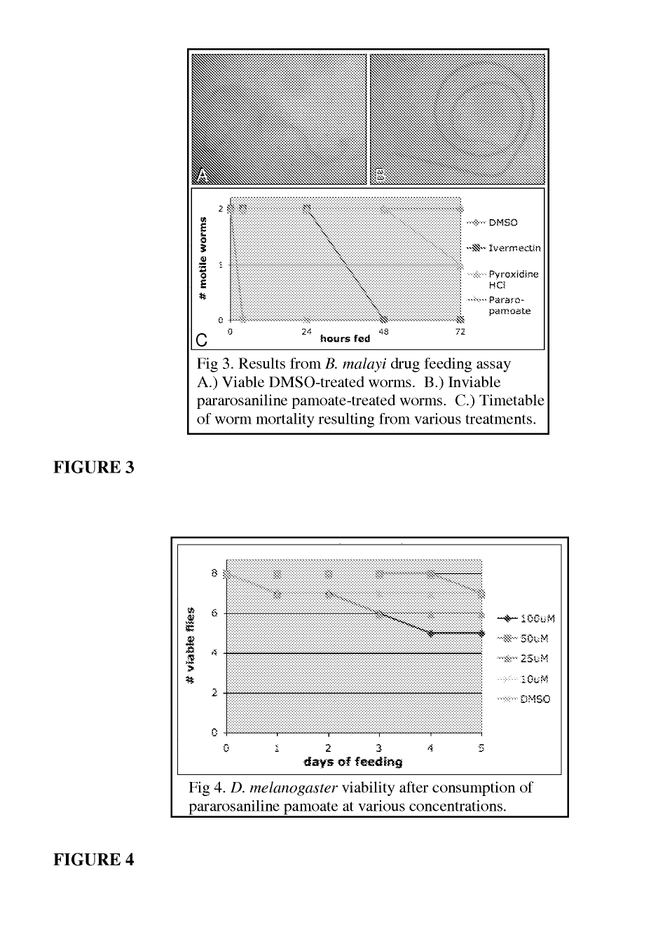

Small molecules of interest found by the primary tissue culture screen must be tested in animal models to determine their relevance to Wolbachia in vivo. Because Wolbachia are vital endosymbionts of Brugia malayi worms that cause Elephantiasis, the next important step was to validate whether compounds found in the high-throughput screen could affect the viability of those worms. To assay this, we performed a pilot feeding assay by distributing Brugia malayi into 12-well plates containing tissue culture media and different compounds added to a final concentration of 100 uM. Two drugs found in the pilot tissue culture screen were used, pararosanaline pamoate and pyroxidine HCl, as well as Ivermectin, a drug currently used against filariasis, and DMSO alone as a control. In this feeding assay, we observed that worms fed with pararosanaline pamoate ceased movement within 2 hours, whereas all other treatments took substantially longer to affect the worms (FIG. 3). Our preliminary dose-response tests of pararosanaline pamoate indicate that the lethal feeding dose lies between 25 uM and 10 uM.

Confocal imaging of untreated worms and worms killed by pararosanaline pamoate indicated no visible difference in the number of Wolbachia carried by each host type, but this is in accord with the rapid death induced by the feeding assay, as there is little time for clearing of dead bacteria from host tissues prior to host mortality.

Drugs of primary interest identified by the screening methods of the invention include Pararosaniline Pamoate, Pyrvinium Pamoate, Clofoctol, and Isoreserpine.

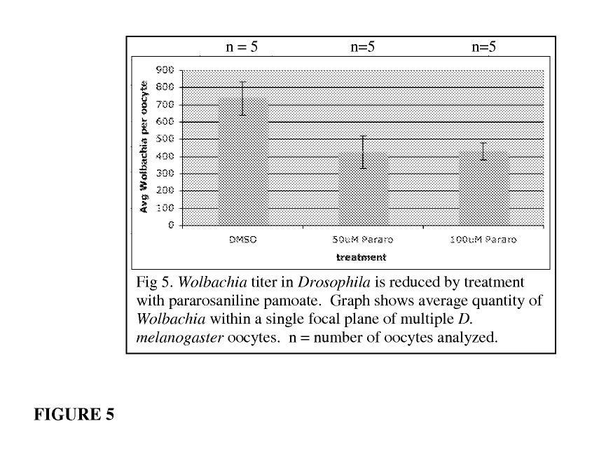

As the next validation step, it was important to determine whether the compounds found in our screen can cure a host of Wolbachia while also permitting host viability. To investigate this, we first performed a feeding assay using Wolbachia-infected Drosophila melanogaster flies. Flies were provided with fly food containing pararosanaline pamoate at concentrations of 100 uM or less, and viability was scored over time. Preliminary results indicate little viability difference between the various drug concentrations and the DMSO control (FIG. 4). To then determine whether the treatment affected the quantity of Wolbachia carried by the D. melanogaster host, we fed pararosaniline pamoate to Wolbachia-infected fruit flies for 3 days and analyzed Wolbachia titer in ovarian tissues. This was performed through use of confocal microscopy and image-based bacterial quantification software. Preliminary results from this test indicated that indeed, Wolbachia titer was reduced to 60% of the control levels upon treatment with pararosaniline pamoate (FIG. 5). This indicates that the pararosaniline pamoate, initially identified by our high throughput screening approach, is an interesting candidate to pursue further as a possible anti-Wolbachia therapeutic drug.

In summary, the invention provides a powerful tool to develop better treatments for Wolbachia-related neglected diseases. We have developed a microscopy-based high throughput screening assay that successfully identifies compounds that reduce Wolbachia titer in tissue culture cells. We demonstrated that these compounds can be successfully validated in animal models by both testing for mortality effects in B. malayi that rely on Wolbachia for survival, and by testing for curative effects in D. melanogaster that carry Wolbachia as a parasite. This study reveals pararosaniline pamoate as a putative fast-acting anti-Wolbachia treatment that may be useful in prevention of Elephantiasis and River Blindness. Follow-up studies will be performed to elucidate the curative effects of pararosaniline pamoate in animal models of Elephantiasis. We have investigated and identified a number of drugs by the screening of the invention that may be used as Wolbachia-depleting, Brugia malayi-killing drugs.

### REFERENCES

Carpenter, A. E. (2007). Image-based chemical screening. Nat Chem Biol 3, 461-465.

Hoerauf, A. (2008). Filariasis: new drugs and new opportunities for lymphatic filariasis and onchocerciasis. Curr Opin Infect Dis 21, 673-681.

Hoerauf, A., et al. (2008). Wolbachia endobacteria depletion by doxycycline as antifilarial therapy has macrofilaricidal activity in onchocerciasis: a randomized placebo-controlled study. Med Microbiol Immunol 197, 295-311.

Karpova, N. (2006). Jupiter, a new Drosophila protein associated with microtubules. Cell Motil Cytoskeleton 63, 301-312.

Perrimon, N., and Mathey-Prevot, B. (2007). Applications of high-throughput RNA interference screens to problems in cell and developmental biology. Genetics 175, 7-16.

Saint Andre, A. et al. (2002). The role of endosymbiotic Wolbachia bacteria in the pathogenesis of river blindness. Science 295, 1892-1895.

Szollosi, A., and Debec, A. (1980). Presence of Rickettsias in Haploid Drosophila melanogaster Cell Lines. Extrait Biologie Cellulaire 38, 129-134.

Turner, J. D., et al. (2009). Wolbachia Lipoprotein Stimulates Innate and Adaptive Immunity through Toll-like Receptors 2 and 6 to Induce Disease Manifestations of Filariasis. J Biol Chem 284, 22364-22378.

1. A pharmaceutical formulation for the prevention and treatment of Wolbachia-related disease, the pharmaceutical formulation containing a drug that kills Wolbachia in vitro and in vivo, wherein the drug is selected from the group consisting of one or more of Pararosaniline Pamoate, Pyrvinium Pamoate, Clofoctol, and Isoreserpine, either singly or in combination.

2. The pharmaceutical formulation of claim 1 wherein the drug consists of Pararosaniline Pamoate.

3. The pharmaceutical formulation of claim 1 wherein the drug consists of Pyrvinium Pamoate.

4. The pharmaceutical formulation of claim 1 wherein the drug consists of Clofoctol.

5. The pharmaceutical formulation of claim 1 wherein the drug consists of Pararosaniline Pamoate, Pyrvinium Pamoate, Clofoctol, and Isoreserpine

6. The pharmaceutical formulation of claim 1 that additionally comprises one or more additives, for example pharmaceutically acceptable excipients, carriers, penetration enhancers, stabilizers, buffers or other materials.

7-9. (canceled)

10. A method for treating of preventing Wolbachia-related disease comprising the following steps: (1) providing an anti-Wolbachia formulation wherein the formulation comprises at least one compound selected from the group consisting of Pararosaniline Pamoate, Pyrvinium Pamoate, Clofoctol, and Isoreserpine; (2) providing a subject in need of treatment or prevention of a Wolbachia-related disease; (3) providing to the subject a therapeutically effective amount of the anti-Wolbachia formulation by various means including (but not limited to) orally, intravenously, intramuscularly, by inhalation, or topically, either in a bolus or a sustained release form wherein such treatment results in the measurable killing of Wolbachia in vivo; and optionally, determining the clinical effect of the treatment.

11. The method of claim 10 wherein the quantity of viable Wolbachia organisms is reduced by at least 40% when compared to the pre-treatment level.

12. A method for the production of pharmaceutical formulations to treat Wolbachia-related disease, the method comprising compounding a pharmaceutically acceptable excipient or carrier with at least one compound selected from the group consisting of Pararosaniline Pamoate, Pyrvinium Pamoate, Clofoctol, and Isoreserpine, and derivatives, metabolites, precursors, pro-drugs and variants thereof.

13. (canceled)
